(19) United States
(12) Patent Application Publication
(10) Pub. No.: US 2022/0054432 A1
(43) Pub. Date: Feb. 24, 2022

(54) COMPOSITION FOR INCREASING RETENTION OF CAROTENOID IN BLOOD

(71) Applicant: ENEOS Corporation, Tokyo (JP)

(72) Inventors: Moe KAWAMURA, Tokyo (JP); Masahiro HAYASHI, Tokyo (JP); Hideaki HARA, Gifu (JP); Shinsuke NAKAMURA, Gifu (JP); Takashi MAOKA, Kyoto (JP)

(73) Assignee: ENEOS Corporation, Tokyo (JP)

(21) Appl. No.: 17/290,992

(22) PCT Filed: Nov. 5, 2019
(86) PCT No.: PCT/JP2019/043216

§ 371 Date: May 3, 2021

(30) Foreign Application Priority Data
Nov. 5, 2018 (JP) 2018-208414

(51) Int. Cl. A61K 31/122; A23L 33/10

(52) U.S. Cl. CPC A61K 31/122; A23L 33/10

(57) ABSTRACT

The present invention provides a novel composition for increasing the retention of a carotenoid in blood. More specifically, a composition for increasing the retention of a carotenoid in blood, the composition comprising one or more asymmetric carotenoids or a pharmaceutically acceptable salt thereof, is used.

### CROSS-REFERENCE TO RELATED APPLICATIONS

This application is based upon and claims the benefit of priority from Japanese Patent Application No. 2018-208414, filed on Nov. 5, 2018; the entire contents of which are incorporated herein by reference.

### TECHNICAL FIELD

The present invention relates to a novel composition for increasing the retention of a carotenoid in blood.

### BACKGROUND ART

Carotenoids are useful natural pigments used as feed additives, food additives, pharmaceuticals, and the like. The carotenoid includes astaxanthin, canthaxanthin, zeaxanthin, β-cryptoxanthin, lycopene, β-carotene, adonirubin, adonixanthin, echinenone, asteroidenone, and 3-hydroxyechinenone, etc., and has been also used as a mixture. Of these, astaxanthin is useful as a feed additive such as a body color improving agent for farmed fishes such as salmons, trouts, and red seabreams, and an egg yolk color improving agent for poultry. Natural astaxanthin is industrially highly valuable as a safe food additive and health food material. Similar to astaxanthin, adonixanthin and adonirubin are expected to be used as feed additives, food additives, pharmaceuticals, and the like.

Furthermore, β-carotene is used as a feed additive, a food additive, a pharmaceutical, and the like; canthaxanthin is used as a feed additive, a food additive, a cosmetic, and the like; and zeaxanthin is used as a food additive, a feed additive, and the like. In addition, lycopene, echinenone, β-cryptoxanthin, 3-hydroxyechinenone, asteroidenone, etc., are also expected to be used as a feed additive, a food material, and the like. As a method for producing these carotenoids, a chemical synthesis method, an extraction method from natural products, a production method by culturing microorganisms, etc., have been known.

On the other hand, regarding the carotenoid, various useful bioactivities including an anti-inflammatory action and an antioxidant action have been reported (Patent Literature 1), and the effects are required to be enhanced. Meanwhile, there had been no reports that the retention of a carotenoid in blood is improved to enhance the effects.

### RELATED ART DOCUMENTS

### Patent Literature

Patent Literature 1: WO 2014/051100

### SUMMARY OF THE INVENTION

This time, the present inventors have found that, among carotenoids, particularly an asymmetric carotenoid shows excellent retention in blood, and that use of the asymmetric carotenoid can remarkably increase the retention of total carotenoids in blood. The present invention is based on such finding.

Therefore, an object of the present invention is to provide a novel composition for increasing the retention of a carotenoid in blood.

The present invention includes the following inventions.

[1] A composition for increasing the retention of a carotenoid in blood, including one or more asymmetric carotenoids or a pharmaceutically acceptable salt thereof.
[2] The composition according to [1], wherein the asymmetric carotenoid includes at least one selected from the group consisting of adonixanthin, adonirubin, asteroidenone, echinenone, 3-hydroxyechinenone, antheraxanthin, fucoxanthin, citranaxanthin, diatoxanthin, diadinoxanthin, flavoxanthin, neoxanthin, and rubixanthin.
[3] The composition according to [1] or [2], wherein the asymmetric carotenoid includes adonixanthin.
[4] The composition according to any one of [1] to [3], wherein the composition is a mixture of a symmetric carotenoid and an asymmetric carotenoid.
[5] The composition according to [4], wherein the symmetric carotenoid includes at least one selected from the group consisting of astaxanthin, zeaxanthin, phytoene, phytofluene, lycopene, β-carotene, canthaxanthin, lutein, crocetin, violaxanthin, and rhodoxanthin.
[6] The composition according to [4] or [5], wherein the symmetric carotenoid includes astaxanthin and the asymmetric carotenoid includes adonixanthin.
[7] The composition according to any one of [1] to [6], wherein the carotenoid is a microorganism-, animal-, or plant-derived substance, or a chemical synthetic product.
[8] The composition according to [7], wherein the microorganism is Paracoccus carotinifaciens.
[9] The composition according to any one of [1] to [8], wherein the content of adonixanthin relative to the total amount of the asymmetric carotenoid is 5% by mass or more.
[10] The composition according to any one of [1] to [9], wherein the content of adonixanthin relative to the total amount of the carotenoid is 2% by mass or more.
[11] The composition according to any one of [1] to [10] for increasing the total amount of a carotenoid delivered into an organ or a tissue of a subject who takes the composition.
[12] The composition according to any one of [1] to [11] for reducing 8-hydroxy-2′-deoxyguanosine or inhibiting the production thereof in a subject who takes the composition.
[13] The composition according to any one of [1] to [12] for inhibiting oxidative stress in a subject who takes the composition.
[14] The composition according to any one of [1] to [13] for anti-aging.
[15] The composition according to any one of [1] to [14], which is a sustained-release preparation.
[16] The composition according to any one of [1] to [15], which is used for a human.
[17] The composition according to any one of [1] to [16], which is a food and drink or a food additive.
[18] The composition according to any one of [1] to [17], which is a functional food or a pharmaceutical.
[19] Use of one or more asymmetric carotenoids or a pharmaceutically acceptable salt thereof in the production of a composition for increasing the retention of a carotenoid in blood.
[20] A method for increasing the retention amount of a carotenoid in blood in a subject, which includes administering or ingesting an effective dose of one or more asymmetric carotenoids or a pharmaceutically acceptable salt thereof to a subject in need thereof.
[21] One or more asymmetric carotenoids or a pharmaceutically acceptable salt thereof for increasing the retention amount of a carotenoid in blood.

According to the present invention, it is possible to remarkably increase the retention of total carotenoids in blood using an asymmetric carotenoid. Moreover, according to the present invention, it is possible to effectively transfer an asymmetric carotenoid into an organ or a tissue. Furthermore, according to the present invention, it is advantageous for reducing 8-hydroxy-2′-deoxyguanosine (8-OHdG) or inhibiting the production thereof.

### BRIEF DESCRIPTION OF THE DRAWINGS

FIG. 1 is a graph showing the concentration of adonixanthin in serum of mice in the adonixanthin administration group and the concentration of astaxanthin in serum of mice in the astaxanthin administration group.

FIG. 2 is a graph showing the proportion of trans-adonixanthin in serum and each organ of mice in the adonixanthin administration group and the proportion of trans-astaxanthin in serum and each organ of mice in the astaxanthin administration group.

FIG. 3 is a graph showing the concentration of adonixanthin in serum of the adonixanthin-administering monkey and the concentration of astaxanthin in serum of the astaxanthin-administering monkey.

FIG. 4 is a graph showing the concentration of 8-OHdG in serum of mice in the adonixanthin administration group, mice in the astaxanthin administration group, and mice in the control administration group.

### DETAILED DESCRIPTION OF THE INVENTION

A composition for increasing the retention in blood of the present invention is characterized in that it includes one or more asymmetric carotenoids or a pharmaceutically acceptable salt thereof. It is a surprising fact that an asymmetric carotenoid including adonixanthin is useful for the retention in blood, as shown in Test Examples 1 to 4 mentioned later.

### Composition for Increasing Retention in Blood

A composition for increasing the retention in blood of the present invention includes one or more asymmetric carotenoids or a pharmaceutically acceptable salt thereof. The composition of the present invention includes an asymmetric carotenoid as an essential component and can be used for increasing the retention amount of a carotenoid in blood. In other words, the asymmetric carotenoid of the present invention has high retention in blood and is likely to be effectively transferred from blood to an organ persistently. Therefore, the composition of the present invention can also be used as a sustained-release preparation from blood to an organ.

### Carotenoid

Carotenoids are generally a compound group composed of a plurality of isoprene units each having five carbon atoms, the units being bonded to each other, and a carotenoid typically has a basic structure composed of eight isoprene units bonded to each other.

The carotenoid may be a noncyclic (hereinafter also referred to as chain) structure, or may be a combination of a chain block and a cyclic block, and is preferably a combination of a chain block and a cyclic block. When the carotenoid is a combination of a chain block and a cyclic block, the number of isoprene units constituting the chain block includes 1 or more, preferably an even number of 2 or more, and more preferably 4. When the carotenoid is a combination of a chain block and a cyclic block, the cyclic block is disposed at, for example, at least one terminal of the chain block, and preferably at both terminals of the chain block. The cyclic block is an atomic group derived from the isoprene unit, and it is preferably derived from at least two or more isoprene units, and may have a hydroxyl group, a carbonyl group, and/or an alkyl group, etc. The chain block and the cyclic block may be connected with a single bond or a double bond or a triple bond.

The carotenoid may be a free form or a fatty acid ester form. Regarding the above-mentioned carotenoid, it is preferable to use a free form in terms of absorbability. The carotenoid may be a stereoisomer such as an optical isomer and a cis-trans isomer. Furthermore, it is preferable to use these carotenoids as an active ingredient.

The carotenoid can be classified into an asymmetric carotenoid and a symmetric carotenoid based on the molecule structure thereof. According to a preferred embodiment of the present invention, the composition of the present invention is a mixture of a symmetric carotenoid and an asymmetric carotenoid. Each of the asymmetric carotenoid and the symmetric carotenoid will be described below.

### (Asymmetric Carotenoid)

The composition for increasing the retention in blood of the present invention includes an asymmetric carotenoid as an essential component. The asymmetric carotenoid means a carotenoid not having the symmetry of the molecule structure. The carotenoid not having the symmetry of the molecule structure means a carotenoid in which the same atoms do not exist at equal distances opposite to each other from the center of the molecule (center of symmetry) of the carotenoid. For example, in the case of a carotenoid in which cyclic blocks are disposed at both terminals of the chain block, the asymmetric carotenoid includes a carotenoid in which each cyclic block represents a different atomic group. Regarding the symmetry of the carotenoid molecule mentioned above, a difference in the position of double bonds in each cyclic block does not impair the symmetry of the molecule. For example, in the case of a carotenoid in which cyclic blocks are disposed at both terminals of the chain block, the same atoms exist at equal distances opposite to each other from the center of the molecule (center of symmetry) of the carotenoid, and each cyclic block has the symmetry of the arrangement of atoms but does not have the symmetry of the position of double bonds (e.g., lutein, etc.), the carotenoid is classified as a symmetric carotenoid not an asymmetric carotenoid.

The asymmetric carotenoid preferably includes an asymmetric carotenoid in which the proportion existing as the trans isomer is higher than the proportion existing as the cis isomer in an organ.

Examples of the asymmetric carotenoid include, but are not particularly limited to, adonixanthin, adonirubin, asteroidenone, echinenone, 3-hydroxyechinenone, antheraxanthin, fucoxanthin, citranaxanthin, diatoxanthin, diadinoxanthin, flavoxanthin, neoxanthin, rubixanthin, and the like, but adonixanthin is preferred.

Adonixanthin (3,3′-dihydroxy-β,β-caroten-4-one, chemical formula: C₄₀H₅₄O₃, molecular weight: 582.869) has a structure represented by the following formula:

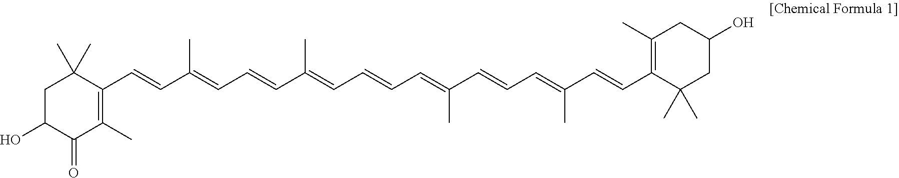 [SMILES: CC1=C(/C=C/C(C)=C/C=C/C(C)=C/C=C/C=C(C)/C=C/C=C(C)/C=C/C2=C(C)C(=O)C(O)CC2(C)C)C(C)(C)CC(O)C1]

Examples of an optical isomer of adonixanthin can include at least one selected from the group consisting of a 3S,3′R-isomer, a 3S,3′S-isomer, a 3R,3′S-isomer, and a 3R,3′R-isomer, and the optical isomer is preferably a 3S,3′R-isomer. A cis-trans isomer of adonixanthin may be a cis isomer, a trans isomer, or a combination thereof. The cis-trans isomer of adonixanthin is preferably a combination of a cis isomer and a trans isomer, or a trans isomer.

Adonirubin (3-hydroxy-β,β-carotene-4,4′-dione, chemical formula: C₄₀H₅₂O₃, molecular weight: 580.853) has a structure represented by the following formula:

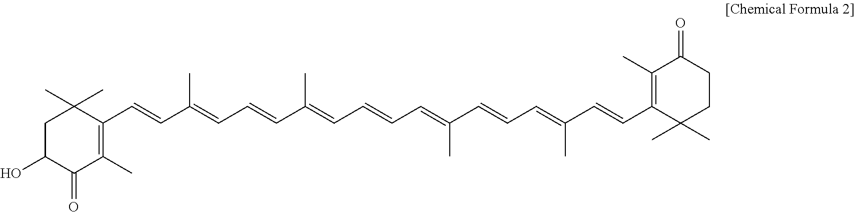 [SMILES: CC1=C(/C=C/C(C)=C/C=C/C(C)=C/C=C/C=C(C)/C=C/C=C(C)/C=C/C2=C(C)C(=O)C(O)CC2(C)C)C(C)(C)CCC1=O]

A cis-trans isomer of adonirubin may be a cis isomer, a trans isomer, or a combination thereof. Examples of a cis isomer can include a 13-cis isomer, and the cis-trans isomer is preferably a trans isomer.

One asymmetric carotenoid may be used alone, or two or more asymmetric carotenoids may be used in combination, but the asymmetric carotenoid preferably includes adonixanthin.

### (Symmetric Carotenoid)

The composition of the present invention may further include a symmetric carotenoid in addition to the above-mentioned asymmetric carotenoid. The symmetric carotenoid means a carotenoid having the symmetry of the molecule structure. The carotenoid having the symmetry of the molecule structure means a carotenoid in which the same atoms exist at equal distances opposite to each other from the center of the molecule (center of symmetry) of the carotenoid. Specifically, in the case of a carotenoid in which cyclic blocks are disposed at both terminals of the chain block, the symmetric carotenoid means a carotenoid in which each cyclic block represents the same atomic group.

The symmetric carotenoid preferably includes a symmetric carotenoid in which the proportion existing as the trans isomer existing is higher than the proportion existing as the cis isomer in an organ.

Examples of the symmetric carotenoid include, but are not particularly limited to, astaxanthin, zeaxanthin, phytoene, phytofluene, lycopene, β-carotene, canthaxanthin, lutein, crocetin, violaxanthin, rhodoxanthin, and the like. Therefore, according to a preferred embodiment of the present invention, the symmetric carotenoid is at least one selected from the group consisting of astaxanthin, zeaxanthin, β-carotene, phytoene, and canthaxanthin.

Astaxanthin (3,3′-dihydroxy-β,β-carotene-4,4′-dione, chemical formula: C₄₀H₅₂O₄, molecular weight: 596.852) is a red pigment and belongs to xanthophyll, which is one of carotenoids, and has a structure represented by the following formula:

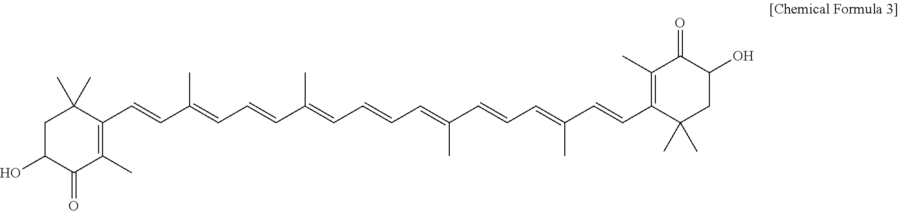 [SMILES: CC1=C(/C=C/C(C)=C/C=C/C(C)=C/C=C/C=C(C)/C=C/C=C(C)/C=C/C2=C(C)C(=O)C(O)CC2(C)C)C(C)(C)CC(O)C1=O]

Examples of an optical isomer of astaxanthin can include at least one selected from the group consisting of a 3S,3′S-isomer, a 3S,3′R-isomer (meso-isomer), and a 3R,3′R-isomer, and the optical isomer is preferably a 3S,3′S-isomer. Astaxanthin may be a cis isomer or a trans isomer of a conjugated double bond in the center of the molecule or a combination thereof. Examples of the cis isomer include a 9-cis isomer, a 13-cis isomer, a 15-cis isomer, a dicis isomer, or a combination thereof. Astaxanthin is preferably a combination of a cis isomer and a trans isomer, or a trans isomer.

Zeaxanthin (β,β-carotene-3,3′-diol, chemical formula: C₄₀H₅₆O₂, molecular weight: 568.87 to 568.89) has a structure represented by the following formula:

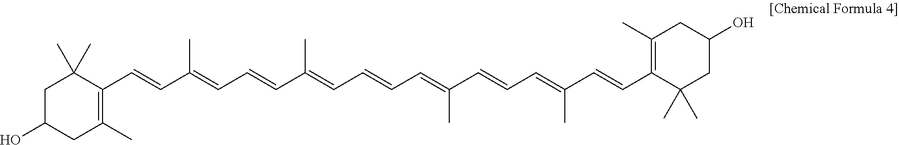 [SMILES: CC1=C(/C=C/C(C)=C/C=C/C(C)=C/C=C/C=C(C)/C=C/C=C(C)/C=C/C2=C(C)CC(O)CC2(C)C)C(C)(C)CC(O)C1]

Examples of an optical isomer of zeaxanthin can include at least one selected from the group consisting of a 3S,3′S-isomer, a 3R,3′S-isomer, and a 3R,3′R-isomer, and the optical isomer is preferably a 3R,3′R-isomer. A cis-trans isomer of zeaxanthin may be a cis isomer, a trans isomer, or a combination thereof. Examples of the cis-trans isomer include an all-trans isomer, a 9-cis isomer, a 13-cis isomer, or a combination thereof. Preferred examples of the stereoisomer include a 3R,3′R-all-trans isomer, a 3R,3′R-9-cis isomer, a 3R,3′R-13-cis isomer, or a combination thereof.

One symmetric carotenoid may be used alone, or two or more symmetric carotenoids may be used in combination, but the symmetric carotenoid preferably includes astaxanthin.

Furthermore, the composition of the present invention is preferably a carotenoid mixture including adonixanthin as the asymmetric carotenoid and including astaxanthin as the symmetric carotenoid. Such carotenoid mixture may further include, in addition to adonixanthin and astaxanthin, an asymmetric carotenoid such as adonirubin, asteroidenone, echinenone, and 3-hydroxyechinenone, and/or a symmetric carotenoid such as zeaxanthin, canthaxanthin, and β-carotene. For example, a carotenoid mixture extracted from a dried bacterial cell of Paracoccus carotinifaciens in accordance with the method mentioned in, e.g., JP 2007-261972 A or JP 2009-50237 A includes adonixanthin, astaxanthin, and adonirubin, and preferably further includes at least one selected from the group consisting of canthaxanthin, asteroidenone, β-carotene, echinenone, 3-hydroxyechinenone, and zeaxanthin.

In the present invention, the carotenoid may be in a form of a pharmaceutically acceptable salt, and these salts are also included in the carotenoid in the present invention. In the present invention, the carotenoid may form a salt with an acid or a base. In the present invention, the pharmaceutically acceptable salt is not particularly limited as long as it forms a pharmaceutically acceptable salt with astaxanthin, adonirubin, adonixanthin, and/or zeaxanthin. Specific example thereof include, but are not limited to, hydrohalides (e.g., hydrofluorides, hydrochlorides, hydrobromates, hydroiodides, etc.), inorganic acid salts (e.g., sulfates, nitrates, perchlorates, phosphates, carbonates, bicarbonates, etc.), organic carboxylates (e.g., acetates, oxalates, maleates, tartrates, fumarates, citrates, etc.), organic sulfonates (e.g., methanesulfonates, trifluoromethanesulfonates, ethanesulfonates, benzenesulfonates, toluenesulfonates, camphorsulfonates, etc.), amino acid salts (e.g., aspartates, glutamates, etc.), quaternary amine salts, alkali metal salts (e.g., sodium salts, potassium salts, etc.), and alkaline earth metal salts (e.g., magnesium salts, calcium salts, etc.), etc.

The carotenoid of the present invention may be a commercially available product, or a chemical synthetic product produced by conventional chemical synthesis methods or a microorganism-, animal-, or plant-derived substance (naturally-derived substance) produced by fermentation methods with microorganisms or extraction and purification from microorganisms, animals, or plants, or the like can be used. Such microorganism includes bacteria, algae, and yeasts. The microorganism-, animal-, or plant-derived substance as used herein is a product obtained from microorganisms, animals, or plants, and preferably may be a genus Paracoccus microorganism-derived substance, and more preferably may be a Paracoccus carotinifaciens-derived substance.

For example, the method for extracting and purifying astaxanthin, adonirubin, and adonixanthin from microorganisms includes the following method. A dried bacterial cell of Paracoccus carotinifaciens is subjected to extraction at room temperature using acetone, followed by concentration of the extract with an evaporator. When the concentrated solution is separated into two layers, a hexane-chloroform (1:1) mixture is added to the concentrate to mix well, followed by a separation operation to obtain an organic solvent layer. The organic solvent layer is concentrated to dryness with an evaporator. The concentrated and dried product is dissolved in chloroform, and each carotenoid is separated with a silica gel column. For example, it is possible to obtain a free form of adonirubin by further purifying a fraction eluted with acetone:hexane (3:7) using HPLC (Shim-pack PRC-SIL (Shimadzu Corporation), acetone:hexane (3:7)). It is also possible to obtain a free form of astaxanthin as a crystal by concentrating a fraction eluted with acetone:hexane (5:5) to allow to stand at 4° C. Furthermore, it is possible to obtain a free form of adonixanthin by further purifying a fraction eluted with acetone using HPLC (Shim-pack PRC-SIL, acetone:hexane (4:6)).

The method for extracting and purifying zeaxanthin from microorganisms includes the following method. It is possible to extract zeaxanthin using a water-soluble organic solvent such as acetone from a precipitated culture or a precipitated dried product of a genus Paracoccus microorganism. Furthermore, it is also possible to further purify zeaxanthin by perform liquid-liquid extraction after adding a nonpolar organic solvent and/or water to the obtained water-soluble organic solvent extract.

As the method for extracting and purifying zeaxanthin, extraction and purification can be performed in accordance with the procedure mentioned in US 2014/0113354 A1. For example, it is possible to obtain zeaxanthin by extracting a culture with a solvent such as acetone, and by eluting the acetone extract with a silica gel column using an ethyl acetate-hexane (3:7) mixture.

The content of the asymmetric carotenoid in the composition of the present invention is not particularly limited as long as the effect of the present invention is not impaired, and, for example, is 0.1 to 99% by mass, preferably 0.1 to 95% by mass, more preferably 0.1 to 90% by mass, and further preferably 0.1 to 85% by mass, based on the total mass of the composition.

The content of adonixanthin in the asymmetric carotenoid of the present invention is not particularly limited, and, for example, is 0.1 to 99% by mass, preferably 1 to 99% by mass, more preferably 3 to 99% by mass, and further preferably 5 to 99% by mass.

The content of adonixanthin in total carotenoids in the composition of the present invention is not particularly limited, and, for example, is 0.1 to 99% by mass, preferably 0.5 to 99% by mass, more preferably 1 to 99% by mass, and further preferably 2 to 99% by mass.

The content of the symmetric carotenoid in the composition of the present invention is not particularly limited as long as the effect of the present invention is not impaired, and, for example, is 0.1 to 99% by mass, preferably 0.1 to 95% by mass, more preferably 0.1 to 90% by mass, and further preferably 0.1 to 85% by mass, based on the total mass of the composition.

The content of adonixanthin, astaxanthin, and adonirubin in the composition of the present invention can be measured by the HPLC method in accordance with the procedure mentioned in Toxicol Rep. 2014 Aug. 25; 1:582-588. The content of zeaxanthin in the composition of the present invention can be measured by the HPLC method in accordance with the procedure mentioned in [Examples] of JP 6132905 B.

The composition of the present invention can be provided as a composition into which, if desired, an orally acceptable or pharmaceutically acceptable additive is formulated together with the above-mentioned carotenoid. Examples of the additive mentioned above include solvents, solubilizing agents, solubilizers, lubricants, emulsifiers, isotonizing agents, stabilizers, preservatives, antiseptics, surfactants, adjusters, chelating agents, pH adjusters, buffers, excipients, thickeners, coloring agents, aromatics, or perfumes.

The composition of the present invention can be prepared by a known method such as mixing, dissolving, dispersing, and suspending the above-mentioned carotenoid and, if desired, an orally acceptable or pharmaceutically acceptable additive. In preparation of the composition of the present invention, a mixture, a dissolved substance, a dispersed substance, a suspension, etc., prepared by the above-mentioned method may be subjected to homogenization treatment or sterilization treatment, as long as the effect of the present invention is not impaired.

The form of the composition of the present invention is not particularly limited as long as the effect of the present invention is not impaired, and may be solid, semi-solid (including paste and gel), or liquid (including oil and slurry), and the form is preferably solid or liquid.

The dosage form of the composition of the present invention is not particularly limited as long as the effect of the present invention is not impaired, and examples thereof include injection, tablet (e.g., plain tablet, sugar-coated tablet, film-coated tablet, enteric-coated tablet, sustained-release tablet, orally disintegrating tablet, sublingual tablet, chewable tablet, etc.), capsule (e.g., hard capsule, soft capsule), elixir, pill, dust, powder, granule, solution, troche, syrup, dry syrup, emulsion, suspension, liquid, inhalant, aerosol agent, powder inhalant, suppository, ointment, cream, gel, patch, poultice, lotion, drop, ophthalmic ointment, eye drop, and nasal drop. The dosage form of the composition of the present invention is preferably a dosage form for oral intake or administration, and examples thereof include tablet, capsule, pill, dust, powder, granule, syrup, dry syrup, emulsion, liquid, suspension, solution, and troche.

A method for administration or intake of the composition of the present invention is not particularly limited, and examples thereof include injection such as infusion, intravenous injection, intramuscular injection, subcutaneous injection, and intradermal injection, and oral, transmucosal, percutaneous, intranasal, intraoral, etc., administration or intake, and the method is preferably oral intake or administration.

Examples of the composition of the present invention include foods and drinks such as foods or drinks, food additives, feeds, pharmaceuticals, quasi drugs, or cosmetics, and foods and drinks are preferred in terms of simpleness of intake.

The food and drink of the present invention may be those obtained by preparing the composition of the present invention as a food and drink as it is, those obtained by further formulating various proteins, saccharides, fats, trace elements, vitamins, plant extracts, or other active ingredients (e.g., bacteria such as lactic acid bacteria and Bacillus bacteria, fungi such as yeasts, dietary fibers, DHA or EPA), those obtained by making the composition of the present invention into liquid (such as solution), semiliquid, or solid, or those obtained by adding the composition of the present invention to a general food and drink.

Specific examples of the above-mentioned food and drink include instant foods such as instant noodles, pre-packaged foods, canned foods, foods for microwave cooking, instant soups and miso soups, and freeze-dried foods; drinks such as soft drinks, fruit juice drinks, vegetable drinks, soy milk drinks, coffee drinks, tea drinks, powdered drinks, concentrated drinks, alcoholic drinks, and jelly drinks; energy drinks; flour products such as breads, pastas, noodles, cake mixes, and bread crumbs; confectionery such as candies, gummies, jellies, caramels, chewing gums, chocolates, cookies, biscuits, cakes, pies, snacks, crackers, Japanese-style confectionery, and dessert confectionery; nutrition bars; sports bars; seasonings such as sauces, processed tomato seasonings, flavor seasonings, cooking mixes, sauces, dressings, soups, and curry or stew mixes; oils and fats such as processed oils and fats, butter, margarine, and mayonnaise; dairy products such as milk-based drinks, yogurts, lactic acid bacteria drinks, ice creams, and creams; processed agricultural products such as agricultural canned foods, jams and marmalades, and cereals; processed meat foods such as hams, bacons, sausages, and roast pork: and frozen foods, but the food and drink is not limited thereto.

The food and drink of the present invention also include health foods, supplements, functional foods (e.g., including foods for specified health uses, nutritional functional foods, or foods with function claims), foods for special dietary uses (e.g., including foods for the sick, infant formulas, powdered milk for pregnant and lactating women, or foods for persons with swallowing/chewing difficulties), or liquid modified milk for infants (also referred to as liquid milk for infants). As mentioned later, since the composition of the present invention has an action to inhibit or treat oxidative stress on an organ or a tissue, or a disease or a symptom attributable thereto, there is provided a food and drink for inhibition or treatment of oxidative stress on an organ or a tissue, or a disease or a symptom attributable thereto. In other words, the food and drink of the present invention can be provided as a food and drink for a human having a disease or a symptom attributable to oxidative stress on an organ or a tissue. Furthermore, foods and drinks such as functional foods may be provided with being labelled with “antioxidant action is expected”, “oxidative stress is reduced”, “for anti-aging”, and the like.

The intake or dose of the composition of the present invention is not particularly limited, and can be determined by considering the prescription of the composition, the type of an asymmetric carotenoid, purity, the type of a subject, age or body weight of a subject, symptoms, the duration of intake or administration, the form of the composition, the method for intake or administration, and the like. The composition of the present invention is preferably composed of a form of daily intake unit so that the effective dose is for inhibition or treatment of oxidative stress on an organ or a tissue, or a symptom attributable thereto. For example, when the composition of the present invention is orally taken, the asymmetric carotenoid can be formulated into the composition so that the intake or dose of one or more asymmetric carotenoids and a pharmaceutically acceptable salt thereof is in the range of 0.01 to 10,000 mg, preferably 0.05 to 1,000 mg, and more preferably 0.1 to 100 mg per day per adult with a body weight of 60 kg. In the present invention, a drug other than the carotenoid used in combination with the asymmetric carotenoid can also be appropriately determined using a clinically used intake or dose, respectively, as a standard.

The daily intake or dose of the composition of the present invention is appropriately selected according to the prescription of the composition, etc. The daily intake or dose of the composition of the present invention may be, for example, taken by or administered to a subject once or plural times, and is preferably taken by or administered to a subject once. Therefore, the daily number of intake or administration of the composition of the present invention includes 1 to 5 times a day, preferably 1 to 3 times a day, and more preferably once a day.

According to one embodiment, a subject to whom the composition of the present invention is applied is not particularly limited as long as the effect of the present invention is not impaired, and is preferably mammals, and more preferably primates such as humans, dogs, and cats. The subject may be healthy subjects (healthy animals) or patients (patient animals).

According to the composition of the present invention, it is possible to reduce 8-OHdG or inhibit the production thereof in a subject who takes the composition. According to the composition of the present invention, it is advantageous in that it is possible to reduce 8-OHdG in blood or inhibit the production thereof. Particularly, an asymmetric carotenoid such as adonixanthin is advantageous in that it is possible to reduce 8-OHdG more or inhibit the production of 8-OHdG more since it has a higher action to increase the retention in blood than that of a symmetric carotenoid such as astaxanthin.

Here, 8-OHdG is known to be one of oxidative stress markers. By using an oxidative stress marker as an index, it is possible to grasp a damaged state or a change therein of an organ or a tissue exposed to free radicals such as reactive oxygen species from an analysis of components such as blood without invasion of the organ or the tissue. 8-OHdG is produced after deoxyguanosine (dG), which is a component of DNA in a cell, is oxidized by a hydroxy radical produced by oxidative stress. Therefore, 8-OHdG is used as an index reflecting high or low oxidative stress.

According to the composition of the present invention, it is possible to inhibit oxidative stress on an organ or a tissue. Therefore, according to the composition of the present invention, it is possible to inhibit or treat oxidative stress on an organ or a tissue, or a disease (disorder) or a symptom attributable thereto. Therefore, according to one embodiment of the present invention, the composition of the present invention is provided as a composition for inhibition or treatment of oxidative stress on an organ or a tissue, or a disease or a symptom attributable thereto. “Inhibition” of a disease or a symptom attributable thereto as used herein includes the meaning of improvement in a disease or a symptom attributable thereto by a non-medical practice, as well as the meaning of “prevention” in which provision is made for expected worsening in advance and occurrence or recurrence of a disease or a symptom attributable thereto is prevented in the bud by a non-medical practice or a medical practice. “Treatment” means improvement in a disease or a symptom attributable thereto by a medical practice. Improvement as used herein includes stopping, alleviating, or delaying the progress or worsening of a disease or a symptom attributable thereto.

Examples of the disease or symptom attributable to oxidative stress on an organ or a tissue mentioned above include, but are not particularly limited to, cranial nerve disease, Alzheimer's disease, Parkinson's disease, schizophrenia, bipolar disorder, fragile X syndrome, amyotrophic lateral sclerosis, polyglutamine disease, prion disease, cerebral infarction, cerebral stroke, hypertension, arteriosclerosis, angina pectoris, heart disease, cancer, chronic fatigue syndrome, aging, sarcopenia, frailty, locomotive syndrome, inflammation, respiratory disease, skin disease, gastrointestinal disease, cataract, diabetes mellitus, and the like.

The composition of the present invention can increase the total amount of a carotenoid delivered into an organ or a tissue of a subject who takes the composition, using an asymmetric carotenoid. Furthermore, the composition of the present invention can increase the retention amount of total carotenoids in blood, thereby gradually transferring the carotenoids into an organ or a tissue. Therefore, according to the other embodiment, regarding the composition of the present invention, there is provided a composition for transferring a carotenoid into an organ or a tissue or for making a carotenoid be retained in an organ or a tissue. Examples of such organ or tissue include brain, heart, lung, spleen, liver, kidney, skin, and the like. Examples of specific regions of the brain include cerebrum (e.g., cerebral cortex, cerebral medulla), cerebellum, midbrain, striatum (e.g., striatum putamen, striatum caudate nucleus), hippocampus, medulla oblongata, diencephalon, and the like. Since the composition of the present invention can be transferred into and/or retained in an organ, it is advantageous for inhibiting or treating a disease related to each organ or a symptom attributable thereto. Examples of such disease related to the brain include Alzheimer's disease, Parkinson's disease, schizophrenia, bipolar disorder, fragile X syndrome, amyotrophic lateral sclerosis, polyglutamine disease, prion disease, cerebral infarction, cerebral stroke, arteriosclerosis, angina pectoris, heart disease, cancer, chronic fatigue syndrome, aging, and the like.

According to the other embodiment of the present invention, there is provided a method for inhibiting or treating oxidative stress on an organ or a tissue of a subject, or a disease or a symptom attributable thereto, or a method for transferring a carotenoid such as the above-mentioned asymmetric carotenoid into an organ or a tissue of a subject, which includes administering or ingesting a composition including an effective dose of one or more asymmetric carotenoids or a pharmaceutically acceptable salt thereof to a subject. According to further the other embodiment of the present invention, there is provided a method for increasing the retention amount of a carotenoid in blood in a subject, or a method for reducing 8-OHdG in a subject or inhibiting the production thereof, which includes administering or ingesting a composition including an effective dose of one or more asymmetric carotenoids or a pharmaceutically acceptable salt thereof to a subject. According to further the other embodiment of the present invention, there is provided a method for inhibiting or treating oxidative stress on an organ or a tissue of a subject, or a disease or a symptom attributable thereto, or a method for transferring a carotenoid such as the above-mentioned asymmetric carotenoid into an organ or a tissue of a subject, which includes administering or ingesting an effective dose of one or more asymmetric carotenoids or a pharmaceutically acceptable salt thereof to a subject in need thereof. According to further the other embodiment of the present invention, there is provided a method for increasing the retention amount of a carotenoid in blood in a subject, or a method for reducing 8-OHdG in a subject or inhibiting the production thereof, which includes administering or ingesting an effective dose of one or more asymmetric carotenoids or a pharmaceutically acceptable salt thereof to a subject in need thereof. “Effective dose” as used herein can be set in the same manner as the content of one or more asymmetric carotenoids or a pharmaceutically acceptable salt thereof, etc., in daily intake unit. The above-mentioned method can also be applied to a subject only by a non-medical practice. Therefore, according to the other embodiment of the present invention, there is provided a method for inhibiting oxidative stress on an organ or a tissue of a subject, or a disease or a symptom attributable thereto, or a method for transferring a carotenoid such as the above-mentioned asymmetric carotenoid into an organ or a tissue of a subject, which includes administering or ingesting a composition including an effective dose of one or more asymmetric carotenoids or a pharmaceutically acceptable salt thereof to a subject (excluding a medical practice, for example, a medical practice for a human). According to further the other embodiment of the present invention, there is provided a method for increasing the retention amount of a carotenoid in blood in a subject, or a method for reducing 8-OHdG in a subject or inhibiting the production thereof, which includes administering or ingesting a composition including an effective dose of one or more asymmetric carotenoids or a pharmaceutically acceptable salt thereof to a subject (excluding a medical practice, for example, a medical practice for a human). According to further the other embodiment of the present invention, there is provided a method for inhibiting or treating oxidative stress on an organ or a tissue of a subject, or a disease or a symptom attributable thereto, or a method for transferring a carotenoid such as the above-mentioned asymmetric carotenoid into an organ or a tissue of a subject, which includes administering or ingesting an effective dose of one or more asymmetric carotenoids or a pharmaceutically acceptable salt thereof to a subject in need thereof (excluding a medical practice, for example, a medical practice for a human). According to further the other embodiment of the present invention, there is provided a method for increasing the retention amount of a carotenoid in blood in a subject, or a method for reducing 8-OHdG in a subject or inhibiting the production thereof, which includes administering or ingesting an effective dose of one or more asymmetric carotenoids or a pharmaceutically acceptable salt thereof to a subject in need thereof (excluding a medical practice, for example, a medical practice for a human). The above-mentioned method of the present invention can be performed in accordance with the content mentioned herein in the composition of the present invention.

According to the other embodiment of the present invention, there is provided use of one or more asymmetric carotenoids or a pharmaceutically acceptable salt thereof for inhibiting or treating oxidative stress on an organ or a tissue, or a disease or a symptom attributable thereto, or transferring a carotenoid such as the above-mentioned asymmetric carotenoid into an organ or a tissue. According to further the other embodiment of the present invention, there is provided use of one or more asymmetric carotenoids or a pharmaceutically acceptable salt thereof for increasing the retention amount of a carotenoid in blood, or reducing 8-OHdG or inhibiting the production thereof.

According to the other embodiment of the present invention, there is provided use of one or more asymmetric carotenoids or a pharmaceutically acceptable salt thereof as a composition for inhibiting or treating oxidative stress on an organ or a tissue, or a disease or a symptom attributable thereto, or transferring a carotenoid such as the above-mentioned asymmetric carotenoid into an organ or a tissue. According to further the other embodiment of the present invention, there is provided use of one or more asymmetric carotenoids or a pharmaceutically acceptable salt thereof as a composition for increasing the retention of a carotenoid in blood, or a composition for reducing 8-OHdG or inhibiting the production thereof.

According to the other embodiment of the present invention, there is provided use of one or more asymmetric carotenoids or a pharmaceutically acceptable salt thereof in the production of a composition for inhibiting or treating oxidative stress on an organ or a tissue, or a disease or a symptom attributable thereto, or transferring a carotenoid such as the above-mentioned asymmetric carotenoid into an organ or a tissue. According to further the other embodiment of the present invention, there is provided use of one or more asymmetric carotenoids or a pharmaceutically acceptable salt thereof in the production of a composition for increasing the retention of a carotenoid in blood, or a composition for reducing 8-OHdG or inhibiting the production thereof.

According to the other embodiment of the present invention, there is provided one or more asymmetric carotenoids or a pharmaceutically acceptable salt thereof for inhibiting or treating oxidative stress on an organ or a tissue, or a disease or a symptom attributable thereto, or transferring a carotenoid such as the above-mentioned asymmetric carotenoid into an organ or a tissue. According to further the other embodiment of the present invention, there is provided one or more asymmetric carotenoids or a pharmaceutically acceptable salt thereof for increasing the retention amount of a carotenoid in blood, or reducing 8-OHdG or inhibiting the production thereof.

Any of the embodiments of use and the compound (asymmetric carotenoid) mentioned above can be performed in accordance with the description on the composition or the method of the present invention.

### EXAMPLES

The present invention will be more specifically described below by way of Preparation Examples and Test Examples, but the technical scope of the present invention is not limited to these Examples. Unless otherwise specified, all percentages and ratios used in the present invention are by mass. Unless otherwise specified, the unit and the measurement methods as used herein are in accordance with the JIS Standard.

### Preparation Example 1: Preparation of Adonixanthin, Adonirubin, and Astaxanthin

In accordance with the method mentioned in JP 2012-158569 A, a free form of astaxanthin, a free form of adonirubin, and a free form of adonixanthin were prepared. The method will be briefly mentioned below.

A dried bacterial cell of Paracoccus carotinifaciens was subjected to extraction at room temperature using acetone. The extract thus obtained was concentrated with an evaporator, and when the concentrated solution was separated into two layers, a hexane-chloroform (1:1) mixture was added to the concentrate to mix well, followed by separation operation to obtain an organic solvent layer.

The organic solvent layer thus obtained was concentrated to dryness with an evaporator. The concentrated and dried product was dissolved in chloroform, and each carotenoid was separated with a silica gel column. Specifically, a fraction eluted with 300 mL of acetone:hexane (3:7) was further purified using HPLC (Shim-pack PRC-SIL (Shimadzu Corporation), acetone:hexane (3:7)) to obtain a free form of adonirubin (hereinafter also simply referred to as adonirubin). A fraction eluted with acetone:hexane (5:5) was concentrated, followed by allowing to stand at 4° C., thus obtaining a free form of astaxanthin as a crystal (hereinafter also simply referred to as astaxanthin). A fraction eluted with acetone was further purified using HPLC (Shim-pack PRC-SIL, acetone:hexane (4:6)) to obtain a free form of adonixanthin (hereinafter also simply referred to as adonixanthin).

### Test Example 1: Confirmation of Retention in Blood and Transfer into Each Organ of Asymmetric Carotenoid in Mice

Adonixanthin was used as an asymmetric carotenoid, and astaxanthin was used as a symmetric carotenoid. As experimental animals, ICR strain mice were used. Fourteen (14) mice were used, and 4 mice each were assigned to the adonixanthin-administration group and the astaxanthin-administration group, and 6 mice were assigned to the control administration group. In grouping, each group was constituted so that the mean body weight in each group was as uniform as possible based on the body weight on the day before initiation of administration.

For each experimental group, blood was collected before initiation of administration of dosing substances.

To each experimental group, each of adonixanthin, astaxanthin, and olive oil (product number 150-00276, manufactured by Wako Pure Chemical Industries, Ltd.) was orally administered. In the adonixanthin-administration group and the astaxanthin-administration group, adonixanthin and astaxanthin at a volume of 50 mg/kg body weight were orally administered once daily for 10 days (the day of initiation of administration of the dosing substances was defined as day 1) using a flexible stomach tube. In the control administration group, olive oil at a volume of 0.05 mL/kg body weight was orally administered once daily for 10 days using a flexible stomach tube.

During the administration period, the mice were fed ad libitum a pellet (CE-2, manufactured by CLEA Japan, Inc.) and tap water, and maintained on a 12-h light/dark cycle, at 23±3° C., with a relative humidity of 50±20%.

Blood was collected 4 hours after the last administration of each dosing substance. Each of the concentration of adonixanthin in serum of blood collected from the adonixanthin-administration group (concentration relative to 1 mL of serum) and the concentration of astaxanthin in serum of blood collected from the astaxanthin-administration group (concentration relative to 1 mL of serum) was measured. Specifically, 2 mL of ethanol was added to 1 mL of serum, and then 5 mL of a diethyl ether:hexane (2:8, v/v) solution was added, followed by stirring. After allowing to stand, the upper layer was taken and filtered through a filter, followed by evaporation to dryness. The residue was dissolved in an acetone:hexane (2:8, v/v) solution, and subjected to HPLC. As the HPLC device, a Hitachi L-6000 intelligent pump and an L-4250 UV-VIS detector were used. The measurement wavelength was 450 nm, and a column of 5 μm Cosmosil 5SL-II (inner diameter of 250×4.6 mm) (manufactured by Nacalai Tesque, Inc.) was used. As the mobile phase, acetone:hexane (2:8, v/v) was used, and measurement was performed at a flow rate of 1.0 mL/min.

In HPLC, the concentration of adonixanthin was measured as a concentration of each of the cis isomer and the trans isomer.

After blood collection after the last administration, the whole body was perfused with a heparinized lactated Ringer's solution under isoflurane anesthesia, followed by collection of the retina, the heart, the lung, the spleen, the liver, and the kidney. The collected organs were immediately frozen with liquid nitrogen, and were stored at −80° C.

Each of the concentration of adonixanthin in each organ collected from the adonixanthin-administration group (concentration relative to the weight of each organ) and the concentration of astaxanthin in each organ collected from the astaxanthin-administration group (concentration relative to the weight of each organ) was measured. Specifically, each organ was homogenized, and extraction was repeated with acetone until no color occurred. Then, filtration through a filter was performed to evaporate acetone, and diethyl ether:hexane (2:8, v/v) was added to the solution to extract a carotenoid. Furthermore, evaporation to dryness was performed, and the residue was dissolved in acetone:hexane (2:8, v/v) and subjected to HPLC. As the HPLC device, a Hitachi L-6000 intelligent pump and an L-4250 UV-VIS detector were used. The measurement wavelength was 450 nm, and a column of 5 μm Cosmosil 5SL-II (inner diameter of 250×4.6 mm) (manufactured by Nacalai Tesque, Inc.) was used. As the mobile phase, acetone:hexane (2:8, v/v) was used, and measurement was performed at a flow rate of 1.0 mL/min.

In HPLC, the concentration of astaxanthin was measured as a concentration of each of the cis isomer and the trans isomer.

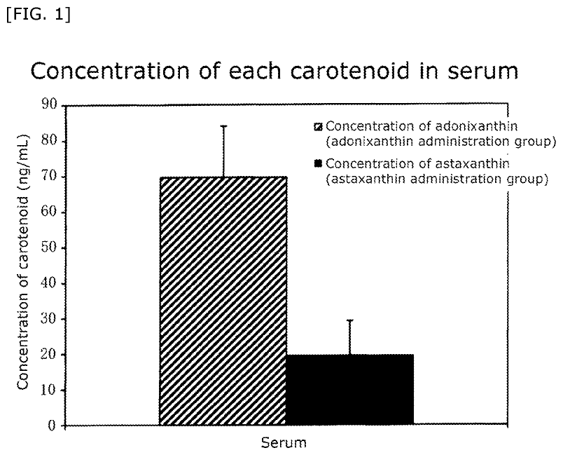

Each of the concentration of adonixanthin in serum collected from the adonixanthin-administration group (total concentration of the cis isomer and the trans isomer) and the concentration of astaxanthin in serum collected from the astaxanthin-administration group (total concentration of the cis isomer and the trans isomer) is shown in FIG. 1. From serum collected from the control administration group, neither adonixanthin nor astaxanthin were detected.

The results of FIG. 1 showed that both of adonixanthin and astaxanthin exist in serum at a high concentration (i.e., are likely to be retained in blood). Particularly, it was shown that adonixanthin, which is an asymmetric carotenoid, is especially likely to be retained in blood.

Each of the concentration of adonixanthin in each organ collected from the adonixanthin-administration group and the concentration of astaxanthin in each organ collected from the astaxanthin-administration group is shown in Table 1. The measured value was expressed as mean. From each organ collected from the control administration group, neither adonixanthin nor astaxanthin were detected.

TABLE 1

| | Organ | | | | |
|---|---|---|---|---|---|
| | Heart | Lung | Spleen | Liver | Kidney |
| Concentration of adonixanthin | 140.9 | 1944.4 | 277.2 | 1926.4 | 773.6 |
| (ng/g) | | | | | |
| (adonixanthin-administration | | | | | |
| group) | | | | | |
| Concentration of astaxanthin | 124.5 | 106.2 | 87.8 | 108.6 | 84.6 |
| (ng/g) | | | | | |
| (astaxanthin-administration | | | | | |
| group) | | | | | |

The results of Table 1 showed that both of adonixanthin and astaxanthin exist in each organ at a high concentration (i.e., are likely to be transferred into each organ and retained therein). Particularly, it was shown that adonixanthin, which is an asymmetric carotenoid, is especially likely to be transferred into each organ and retained therein.

### Test Example 2: Confirmation of Proportion of Trans Carotenoid in Serum and Each Organ of Mice

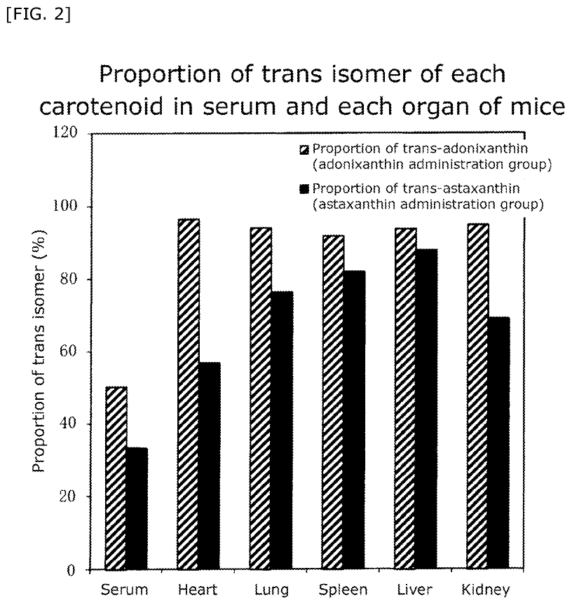

Regarding each of adonixanthin and astaxanthin in serum and each organ of mice of Test Example 1, the proportion of the trans isomer was confirmed. The results are shown in FIG. 2.

The results of FIG. 2 showed that, regarding both of adonixanthin and astaxanthin, the proportion of the trans isomer in each organ is higher than the proportion of the cis isomer. Particularly, it was shown that, regarding adonixanthin, which is an asymmetric carotenoid, the proportion of the trans isomer in each organ is especially higher than the proportion of the cis isomer.

### Preparation Example 2: Preparation of Adonixanthin Dosing Solution and Astaxanthin Dosing Solution

Each of adonixanthin and astaxanthin obtained in Preparation Example 1 was weighed, and olive oil was added to suspend, followed by adjustment so that each concentration was 10 mg/mL, thus obtaining an astaxanthin dosing solution and an adonixanthin dosing solution. Each dosing solution was prepared before use, and stored protected from light on ice until administration.

### Test Example 3: Confirmation of Retention in Blood and Transfer into Each Organ of Asymmetric Carotenoid in Cynomolgus Monkeys

As experimental animals, cynomolgus monkeys were used. Two cynomolgus monkeys were used, and an adonixanthin dosing solution was administered to one monkey (adonixanthin-administering monkey), while an astaxanthin dosing solution was administered to the other monkey (astaxanthin-administering monkey). As the dosing solutions, the dosing solutions obtained in Preparation Example 2 were used, and the dosing solutions at a dose such that the dose of adonixanthin or astaxanthin was 50 mg/kg body weight were administered once daily for 10 days (the day of initiation of administration of the dosing solutions was defined as day 1). As the administration method, a disposable catheter was inserted from the nasal cavity into the stomach, and the dosing solutions were injected into the stomach using a syringe. When the dosing solutions were collected into a syringe, the dosing solutions were collected while being stirred with a stirrer. The dose in each administration was calculated based on the latest body weight at each administration time point (the body weight was measured on the day of initiation of acclimation, the day of end of acclimation, the day of initiation of administration, and before administration on day 8 of administration using an electric balance (HP-40K or GP-40K, both of which are manufactured by A&D Company, Limited)). The administration time was 8:30 to 13:30.

Regarding each cynomolgus monkey, blood was collected before initiation of administration of the dosing solution and 4 hours after the last administration to obtain serum. Specifically, about 30 mL of blood was collected from the femoral vein of each cynomolgus monkey, and the blood was allowed to stand at room temperature for 20 to 60 minutes, followed by centrifugation (room temperature, 1,700×g) for 10 minutes to obtain serum (about 10 mL). The serum thus obtained was stored in a deep freezer (−70° C. or lower).

During the administration period of the dosing solution, each cynomolgus monkey was fed about 108 g (about 12 g×9 pieces) of a pellet once daily at 14:00 to 16:00, and the feed remained by the feeding on the next day (for the day of administration, before administration) was recovered. Each cynomolgus monkey was fed tap water at libitum, and maintained on a 12-h light/dark cycle, at 2±3° C., with a relative humidity of 50±20%.

Regarding each serum 4 hours after the last administration of the dosing solutions, each of the concentration of adonixanthin in serum collected from the adonixanthin-administering monkey and the concentration of astaxanthin in serum collected from the astaxanthin-administering monkey was measured. Specifically, 2 mL of ethanol was added to 1 mL of serum, and then 5 mL of a diethyl ether:hexane (2:8, v/v) solution was added, followed by stirring. After allowing to stand, the upper layer was taken and filtered through a filter, followed by evaporation to dryness. The residue was dissolved in an acetone:hexane (2:8, v/v) solution, and subjected to HPLC. As the HPLC device, a Hitachi L-6000 intelligent pump and an L-4250 UV-VIS detector were used. The measurement wavelength was 450 nm, and a column of 5 μm Cosmosil 5SL-II (inner diameter of 250×4.6 mm) (manufactured by Nacalai Tesque, Inc.) was used. As the mobile phase, acetone:hexane (2:8, v/v) was used, and measurement was performed at a flow rate of 1.0 mL/min.

After blood collection after the last administration of the dosing solutions, an aqueous solution of pentobarbital sodium (manufactured by Tokyo Chemical Industry Co., Ltd.) (64.8 mg/mL) was administered into the cephalic vein at a volume of 0.4 mL/kg body weight to perform anesthesia. After the body weight was measured, each cynomolgus monkey was euthanized by exsanguination, and the brain (cerebral cortex, cerebral medulla, cerebellum, midbrain, striatum putamen, striatum caudate nucleus, hippocampus, medulla oblongata, diencephalon), the heart, the spleen, the liver, the kidneys (right and left), and the retina were collected. Each organ thus collected was stored in a deep freezer (−70° C. or lower).

Each of the concentration of adonixanthin in each organ collected from the adonixanthin-administering monkey (concentration relative to the weight of each organ) and the concentration of astaxanthin in each organ collected from the astaxanthin-administering monkey (concentration relative to the weight of each organ) was measured. Specifically, each organ was homogenized, and extraction was repeated with acetone until no color occurred. Then, filtration through a filter was performed to evaporate acetone, and diethyl ether:hexane (2:8, v/v) was added to the solution to extract a carotenoid. Furthermore, evaporation to dryness was performed, and the residue was dissolved in acetone:hexane (2:8, v/v) and subjected to HPLC. As the HPLC device, a Hitachi L-6000 intelligent pump and an L-4250 UV-VIS detector were used. The measurement wavelength was 450 nm, and a column of 5 μm Cosmosil 5SL-II (inner diameter of 250×4.6 mm) (manufactured by Nacalai Tesque, Inc.) was used. As the mobile phase, acetone:hexane (2:8, v/v) was used, and measurement was performed at a flow rate of 1.0 mL/min.

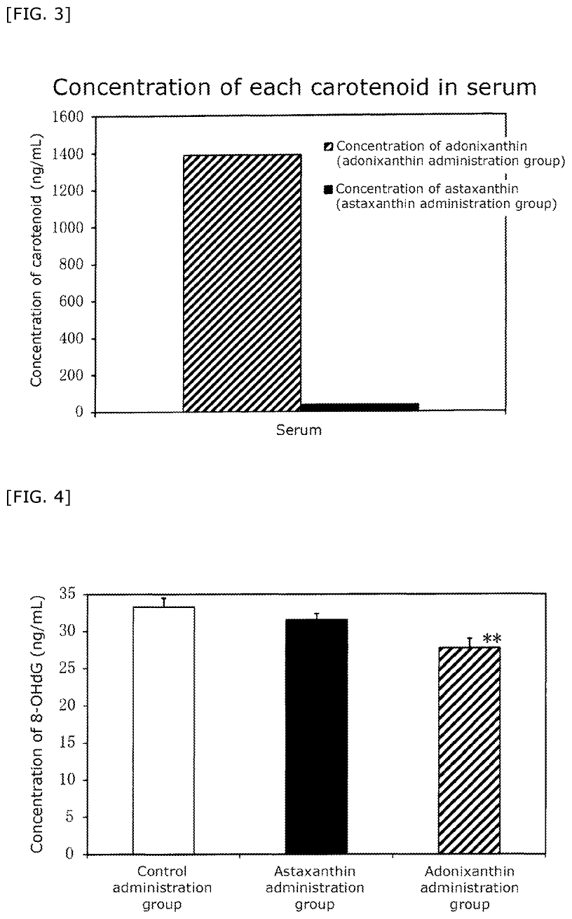

Each of the concentration of adonixanthin in serum collected from the adonixanthin-administering monkey and the concentration of astaxanthin in serum collected from the astaxanthin-administering monkey is shown in FIG. 3.

The results of FIG. 3 showed that both of adonixanthin and astaxanthin exist in serum at a high concentration (i.e., are likely to be retained in blood). Particularly, it was shown that adonixanthin, which is an asymmetric carotenoid, is especially likely to be retained in blood.

Each of the concentration of adonixanthin in each organ collected from the adonixanthin-administering monkey and the concentration of astaxanthin in each organ collected from the astaxanthin-administering monkey is shown in Table 2.

TABLE 2

| | Organ | | | | | | | |
|---|---|---|---|---|---|---|---|---|
| | Brain | | | | | | | |
| | | | | | | Striatum | | |
| | Cerebral | Cerebral | | | Striatum | Caudate | | Medulla |
| | cortex | medulla | Cerebellum | Midbrain | Putamen | nucleus | Hippocampus | oblongata |
| Concentration of | 48.9 | 53.2 | 57.5 | 7.4 | 42.1 | 59.8 | 64.0 | 27.6 |
| adonixanthin (ng/g) | | | | | | | | |
| (adonixanthin- | | | | | | | | |
| administration | | | | | | | | |
| group) | | | | | | | | |
| Concentration of | 4.8 | 8.3 | 14.4 | 2.9 | 5.0 | 10.2 | 7.1 | 8.3 |
| astaxanthin (ng/g) | | | | | | | | |
| (astaxanthin- | | | | | | | | |
| administration | | | | | | | | |
| group) | | | | | | | | |

| | Organ | | | | | | |
|---|---|---|---|---|---|---|---|
| | Brain | | | | Kidney | Kidney | |
| | Diencephalon | Heart | Spleen | Liver | (right) | (left) | Retina |
| Concentration of | 77.1 | 305.4 | 5280.6 | 1124.1 | 1532.0 | 1599.2 | 688.8 |
| adonixanthin (ng/g) | | | | | | | |
| (adonixanthin- | | | | | | | |
| administration | | | | | | | |
| group) | | | | | | | |
| Concentration of | 5.2 | 15.7 | 88.8 | 37.8 | 231.1 | 228.3 | 24.4 |
| astaxanthin (ng/g) | | | | | | | |
| (astaxanthin- | | | | | | | |
| administration | | | | | | | |
| group) | | | | | | | |

The results of Table 2 showed that both of adonixanthin and astaxanthin exist in each organ at a high concentration (i.e., are likely to be transferred into each organ and retained therein). Particularly, it was shown that adonixanthin, which is an asymmetric carotenoid, is especially likely to be transferred into each organ and retained therein.

### Preparation Example 3: Preparation of Adonixanthin Dosing Solution and Astaxanthin Dosing Solution

Each of adonixanthin and astaxanthin obtained in Preparation Example 1 was weighed, and olive oil was added to suspend, followed by adjustment so that the final concentration was 30 mg/mL, thus obtaining an astaxanthin dosing solution and an adonixanthin dosing solution. Each dosing solution was prepared before use.

### Test Example 4: Measurement of 8-OHdG in Blood in Mice

Adonixanthin was used as an asymmetric carotenoid, and astaxanthin was used as a symmetric carotenoid. As experimental animals, ICR strain mice were used. Thirty (30) mice were used, and 10 mice each were assigned to the adonixanthin-administration group, the astaxanthin-administration group, and the control administration group.

In the adonixanthin-administration group and the astaxanthin-administration group, the dosing solutions obtained in Preparation Example 3 were used, and the dosing solutions at a volume (10 mL/kg body weight) such that the dose of adonixanthin or astaxanthin was 300 mg/kg body weight were orally administered once daily for 14 days (the day of initiation of administration of the dosing solutions was defined as day 1). In the control administration group, olive oil at a volume of 10 mL/kg body weight was orally administered once daily for 14 days. As the administration method, administration was performed using a polypropylene disposable syringe and a stomach tube for mice.

During the administration period, the mice were fed ad libitum a pellet (CRF-1, manufactured by Oriental Yeast Co., Ltd.) and tap water, and maintained on a 12-h light/dark cycle, at 21.5 to 24.6° C., with a relative humidity of 52 to 71%.

After end of administration for 14 days, blood was collected from the postcava of the mice under isoflurane absorption anesthesia. The blood thus collected was allowed to stand at room temperature for about 30 minutes, followed by centrifugation (1,500×g, 10 minutes, 4° C.) to collect serum, and the serum was frozen-stored at −80° C. Thereafter, 8-OHdG of the collected serum was measured using 8-hydroxy 2 deoxyguanosine ELISA Kit (ab201734) (manufactured by Abcam plc.) as an ELISA kit for measurement of 8-OHdG.

Each of the concentration of 8-OHdG in serum collected from the adonixanthin-administration group, and the concentration of 8-OHdG in serum collected from the astaxanthin-administration group, and the concentration of 8-OHdG in serum collected from the control administration group is shown in FIG. 4. The concentration was expressed as mean±standard error. Regarding comparison of the concentration of 8-OHdG in blood in the control administration group with that in the other test groups, the difference in mean was tested using Student's t-test (one-sided test) (**: p<0.01 vs control administration group).

The results of FIG. 4 showed that the concentration of 8-OHdG in blood is low in both of the adonixanthin-administration group and the astaxanthin-administration group, and that adonixanthin and astaxanthin reduce 8-OHdG or inhibit the production thereof. Particularly, it was shown that the concentration of 8-OHdG in blood in the adonixanthin-administration group (adonixanthin is an asymmetric carotenoid) is significantly lower than that in the control administration group, and that adonixanthin reduces 8-OHdG more or inhibit the production thereof more.

1.-18. (canceled)

19. A method for increasing the retention amount of a carotenoid in blood in a subject, which comprises administering or ingesting an effective dose of one or more asymmetric carotenoids or a pharmaceutically acceptable salt thereof to a subject in need thereof.

20. (canceled)

21. The method according to claim 19, wherein the asymmetric carotenoid comprises at least one selected from the group consisting of adonixanthin, adonirubin, asteroidenone, echinenone, 3-hydroxyechinenone, antheraxanthin, fucoxanthin, citranaxanthin, diatoxanthin, diadinoxanthin, flavoxanthin, neoxanthin, and rubixanthin.

22. The method according to claim 19, wherein the asymmetric carotenoid comprises adonixanthin.

23. The method according to claim 19, which comprises administering or ingesting the asymmetric carotenoid together with an effective dose of one or more symmetric carotenoids or a pharmaceutically acceptable salt thereof.

24. The method according to claim 23, wherein the symmetric carotenoid comprises at least one selected from the group consisting of astaxanthin, zeaxanthin, phytoene, phytofluene, lycopene, β-carotene, canthaxanthin, lutein, crocetin, violaxanthin, and rhodoxanthin.

25. The method according to claim 23, wherein the symmetric carotenoid comprises astaxanthin and the asymmetric carotenoid comprises adonixanthin.

26. The method according to claim 22, wherein the content of adonixanthin relative to the total amount of the asymmetric carotenoid is 5% by mass or more.

27. The method according to claim 22, wherein the content of adonixanthin relative to the total amount of the carotenoid is 2% by mass or more.

28. The method according to claim 19, wherein the asymmetric carotenoid is in the form of a sustained-release preparation.

29. The method according to claim 19, wherein the asymmetric carotenoid is in the form of food and drink or a food additive.

30. The method according to claim 19, wherein the asymmetric carotenoid is in the form of functional food or a pharmaceutical.

31. A method for increasing the total amount of a carotenoid delivered into an organ or a tissue of a subject, which comprises administering or ingesting an effective dose of one or more asymmetric carotenoids or a pharmaceutically acceptable salt thereof to a subject in need thereof.

32. A method for inhibiting or treating oxidative stress on an organ or a tissue of a subject, or a disease or a symptom attributable thereto, which comprises administering or ingesting an effective dose of one or more asymmetric carotenoids or a pharmaceutically acceptable salt thereof to a subject in need thereof.

33. The method according to claim 32, the oxidative stress can be detected by 8-OHdG.

34. The method according to claim 22, which comprises administering or ingesting the asymmetric carotenoid together with an effective dose of one or more symmetric carotenoids or a pharmaceutically acceptable salt thereof.

35. The method according to claim 34, wherein the symmetric carotenoid comprises at least one selected from the group consisting of astaxanthin, zeaxanthin, phytoene, phytofluene, lycopene, β-carotene, canthaxanthin, lutein, crocetin, violaxanthin, and rhodoxanthin.

36. The method according to claim 35, wherein the content of adonixanthin relative to the total amount of the asymmetric carotenoid is 5% by mass or more.

37. The method according to claim 36, wherein the content of adonixanthin relative to the total amount of the carotenoid is 2% by mass or more.

38. The method according to claim 37, wherein the asymmetric carotenoid is in the form of a sustained-release preparation.

39. The method according to claim 37, wherein the asymmetric carotenoid is in the form of food and drink, a food additive, a functional food, or a pharmaceutical.
